(19) United States
(12) Patent Application Publication
(10) Pub. No.: US 2025/0381554 A1
(43) Pub. Date: Dec. 18, 2025

(54) NOVEL CARBOXY LIGANDS AND USE THEREOF IN CATALYSTS

(71) Applicant: BASF SE, Ludwigshafen am Rhein (DE)

(72) Inventors: Fabian DIELMANN, Innsbruck (DE); Emre LEVENT, Ludwigshafen am Rhein (DE)

(73) Assignee: BASF SE, Ludwigshafen am Rhein (DE)

(21) Appl. No.: 18/878,315

(22) PCT Filed: Jun. 22, 2023
(86) PCT No.: PCT/EP2023/067014

§ 371 Date: Dec. 23, 2024

(30) Foreign Application Priority Data
Jun. 24, 2022 (EP) 22181007.0

(51) Int. Cl. B01J 23/18; B01J 31/04; B01J 37/08; C07D 233/50

(52) U.S. Cl. CPC B01J 23/18; B01J 31/04; B01J 37/08; C07D 233/50

(57) ABSTRACT

The invention relates to a carboxy ligand such as those defined via the general formula (I) specified in the following text. The invention additionally relates to a bismuth-containing catalyst such as those according to the general formula (II), wherein the bismuth-containing catalyst comprises carboxy ligands of the general formula (I). The invention additionally relates to a method for producing such a carboxy ligand and to a method for producing such a bismuth-containing catalyst. During the production of the carboxy ligand according to the invention, a precursor of a ligand according to the general formula (III) is likewise obtained according to the invention. The invention also relates to the use of such a bismuth-containing catalyst for producing compounds which contain a urethane group.

The invention relates to a carboxy ligand as such, as defined by the general formula (I) detailed in the text that follows. The invention further relates to a bismuth catalyst as such according to the general formula (II), where the bismuth catalyst comprises 3 carboxy ligands of the general formula (I). The present invention further relates to a process for preparing such a carboxy ligand and to a process for preparing such a bismuth catalyst. The inventive preparation of the carboxy ligand additionally affords a likewise inventive precursor of a ligand of the general formula (III). The present invention further relates to the use of such a bismuth catalyst for preparation of compounds comprising a urethane group.

WO 2020/160939 relates to a bismuth catalyst as such, as defined by the general formula (I) therein. The bismuth catalyst according to WO 2020/160939 comprises at least one R¹ radical which, according to the general formula (II), comprises a carboxyl fragment where a first carbon atom (α-carbon) is bonded to the carbon atom of the carboxyl group and is in turn directly substituted by at least one aromatic system. WO 2020/160939 further relates to a process for preparing a bismuth catalyst of this kind and to the use of such a bismuth catalyst for preparing compounds comprising a urethane group.

WO 2018/069018 relates to a coating composition system comprising components (A) to (C), and optionally further components. Component (A) is at least one polyhydroxyl group-containing compound, and component (B) is at least one polyisocyanate-containing compound. In contrast, the component (C) is a catalyst comprising at least two salts of an aliphatic monocarboxylic acid having at least 4 carbon atoms. The metal component of the first salt here is bismuth (Bi), while the second salt includes magnesium (Mg), sodium (Na), potassium (K) or calcium (Ca) as metal component. The coating composition systems according to WO 2018/069018 may be configured according to a first option such that all components are present separately from one another, i.e. the individual components are not mixed with one another, whereas, according to a second option of the corresponding coating composition system, the respective components may also be fully or at least partly mixed with one another.

The preparation of compounds comprising a urethane group (urethane bond) has likewise been known for a long time. A compound having a urethane group is generally obtained if a compound having an isocyanate group is reacted with a compound having a hydroxyl group. The reaction generally takes place in the presence of a catalyst. Although tin catalysts show very high activity in such reactions, the use of such tin catalysts, especially alkyltin compounds, should be avoided owing to their (very high) toxicity.

It was therefore an object of the present invention to provide a novel ligand and/or a catalyst comprising such a novel ligand, where the catalyst can be used for preparation of compounds comprising a urethane group.

The object is achieved by a ligand of the general formula (I)

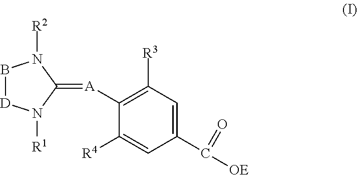

in which the variables are defined as follows:
A is N or (NH)⁺(X)⁻,
B is CH₂, CH, CR⁵ or CR⁵H,
D is CH₂, CH, CR⁶ or CR⁶H,
E is H or alkali metal,
R¹ is H or C₁-C₁₀-alkyl,
R² is H or C₁-C₁₀-alkyl,
R³ is H or C₁-C₁₀-alkyl,
R⁴ is H or C₁-C₁₀-alkyl,
R⁵ is C₁-C₁₀-alkyl,
R⁶ is C₁-C₁₀-alkyl, or R⁵ and R⁶ together form a bridge having four carbon atoms, where B, D, R⁵ and R⁶ may together form a benzene fragment that may optionally be at least monosubstituted by one R⁷ radical,
R⁷ is C₁-C₁₀-alkyl,
X is BF₄, OH or halogen,
   where
   A is (NH)⁺(X)⁻,
   B is CH₂, CH, CR⁵ or CR⁵H,
   D is CH₂, CH, CR⁶ or CR⁶H,
   E is H,
   R¹ is C₂-C₄-alkyl,
   R² is C₂-C₄-alkyl,
   R³ is H or methyl,
   R⁴ is H or methyl,
   R⁵ and R⁶ together form a bridge having four carbon atoms unless B and D are CH₂ or CH, in which case B, D, R⁵ and R⁶ together preferably form an unsubstituted benzene fragment,
   X is BF₄.

The object is also achieved by a bismuth catalyst of the general formula (II)

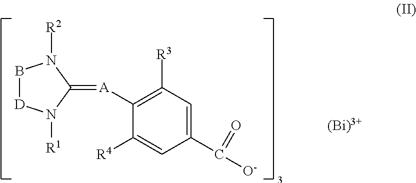

in which the variables are defined as follows:
A is N or (NH)⁺(X)⁻,
B is CH₂, CH, CR⁵ or CR⁵H,
D is CH₂, CH, CR⁶ or CR⁶H,
R¹ is H or C₁-C₁₀-alkyl,
R² is H or C₁-C₁₀-alkyl,
R³ is H or C₁-C₁₀-alkyl,
R⁴ is H or C₁-C₁₀-alkyl,
R⁵ is C₁-C₁₀-alkyl,
R⁶ is C₁-C₁₀-alkyl, or R⁵ and R⁶ together form a bridge having four carbon atoms,
where B, D, R⁵ and R⁶ may together form a benzene fragment that may optionally be at least monosubstituted by one R⁷ radical,
R⁷ is C₁-C₁₀-alkyl,
X is BF₄, OH or halogen,
where, in each of the three ligands of the bismuth catalyst, the respective variables may be defined independently of the respective two other ligands, or all three ligands have the same definition.

One feature of the bismuth catalysts of the invention is that the use of toxic tin catalysts in the preparation of compounds comprising a urethane group can be avoided. The bismuth catalysts of the invention have a comparable catalyst activity to known representatives of the effective tin catalysts, which on the one hand are catalytically active, but on the other hand toxic.

In addition, it has been found that, surprisingly, the use of the inventive bismuth catalyst of the general formula (II) in urethane reactions, through the use of the inventive ligand of the general formula (I), results in occurrence of inhibited progression of the reaction in the first few minutes. This is of particular interest for applications that require inhibition at room temperature. This blocking of the catalyst can lead to longer pot lives in PU applications. What is meant by pot life in accordance with the invention is the length of time for which reactive materials can be processed in the mixed state without observation of losses with regard to quality in the corresponding application. Pot life is determined via gel time.

In addition, it is also not a requirement in the case of the bismuth catalysts of the invention that the catalyst must take the form of a salt in the corresponding acid. The bismuth catalysts of the invention can thus be used even without the presence of the corresponding acid in order to form compounds having urethane groups with high catalytic activity.

From a scientific point of view, it is acceptable, rather than the salt notation used in the context of the present application, to choose a notation/representation for the inventive bismuth catalysts of the general formula (II) where a chemical bond is formed in full or at least in part in each case between the central bismuth atom and the three ligands of general formula (II). In other words, this means that the central bismuth atom does not take the form of a positively charged cation and the corresponding ligands also do not take the form of negatively charged anions; instead, the corresponding charge forms a chemical bond between the corresponding ligands on the one hand and the central bismuth atom on the other hand. In the context of the present invention, the disclosed bismuth catalysts of the invention are therefore also described by such a definition that is not based on a salt.

In the context of the present invention, definitions such as C₁-C₁₀-alkyl, as defined, for example, for the R¹ radical in formula (I) above, mean that this substituent (radical) is an alkyl radical having a carbon atom number of 1 to 10, not including any substituents present in the carbon atom number. The alkyl radical may be either linear or branched, and optionally cyclic. Alkyl radicals having both a cyclic and a linear component are likewise covered by this definition. The same applies to other alkyl radicals such as a C₁-C₆-alkyl radical or a C₁-C₁₂-alkyl radical for example. Examples of alkyl radicals are methyl, ethyl, n-propyl, sec-propyl, n-butyl, sec-butyl, isobutyl, 2-ethylhexyl, tertiary butyl (tert-Bu/t-Bu), pentyl, hexyl, heptyl, cyclohexyl, octyl, nonyl or decyl.

In the context of the present invention, the term “halogen”, as defined for example for the X radical in formula (I) above, means that the substituent (radical) is fluorine, chlorine, bromine or iodine; X is preferably fluorine or chlorine, more preferably chlorine.

In the context of the present invention, the expression “unsubstituted or at least mono-C₁-C₁₀-alkyl-substituted benzene”, as defined in conjunction with the R⁷ radical in formula (I), means that benzene may either be in unsubstituted form or has at least one C₁-C₁₀-alkyl substituent (monosubstituted). If one or more substituents are present, for example disubstituted (trisubstituted or even higher substituted), the appropriate substituents are selected independently of one another from the substituent groups specified in each case.

Given that an appropriate radical, such as R¹ for example, owing to the definition of, for example, formula (II) (in conjunction with formula (I)), may occur twice or more, the individual R¹ radicals may be selected entirely independently according to the respective definitions. Unless otherwise stated in the text that follows, this logically also applies to all other radicals, such as R², R³, R⁵ and/or R⁶.

Unless otherwise specified in the following description, the preferred definitions of the R¹ to R⁷ radicals are in each case the respective unsubstituted definitions.

The present invention is further specificized hereinafter.

The present invention firstly provides a ligand of the general formula (I)

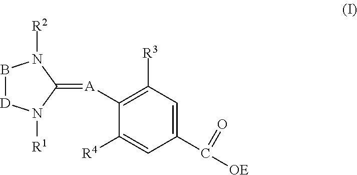

in which the variables are defined as follows:
A is N or (NH)⁺(X)⁻,
B is CH₂, CH, CR⁵ or CR⁵H,
D is CH₂, CH, CR⁶ or CR⁶H,
E is H or alkali metal,
R¹ is H or C₁-C₁₀-alkyl,
R² is H or C₁-C₁₀-alkyl,
R³ is H or C₁-C₁₀-alkyl,
R⁴ is H or C₁-C₁₀-alkyl,
R⁵ is C₁-C₁₀-alkyl,
R⁶ is C₁-C₁₀-alkyl, or R⁵ and R⁶ together form a bridge having four carbon atoms,
where B, D, R⁵ and R⁶ may together form a benzene fragment that may optionally be at least monosubstituted by one R⁷ radical,
R⁷ is C₁-C₁₀-alkyl,
X is BF₄, OH or halogen.

It will be clear to a person skilled in the art that, in the case that R⁵ and R⁶ together form a bridge having four carbon atoms, where B, D, R⁵ and R⁶ together form a benzene fragment, B and D are defined as follows:
B is CR⁵ and
D is CR⁶,
where the R⁵ and R⁶ radicals have a total of 4 carbon and 4 hydrogen atoms, and there are six delocalized π electrons between the total of 6 carbon atoms. In other words, the R¹, R², R⁴, R⁵, B and D radicals form a benzimidazole radical.

In addition, it is also possible that a double bond is present rather than a single bond between B and D. In this case:
B is CH or CR⁵ and
D is CH or CR⁶.

It is preferable in accordance with the invention that, in the general formula (I),
i) B and D, and optionally R⁵ and R⁶, each have the same definition, where B, D, R⁵ and R⁶ together optionally form a benzene fragment which is preferably unsubstituted, and/or
ii) R¹ and R² each have the same definition, and/or
iii) R³ and R⁴ each have the same definition, and/or
iv) alkali metal is sodium or potassium, preferably sodium, if E is alkali metal, and/or
v) halogen is chlorine or bromine, if X is halogen.

However, it is also possible that R¹ and R² are different and/or R³ and R⁴ are different. It is of course also possible that B and D are different.

It is more preferable in accordance with the invention that, in the general formula (I),
A is (NH)⁺(X)⁻,
B is CH₂, CH, CR⁵ or CR⁵H,
D is CH₂, CH, CR⁶ or CR⁶H,
E is H,
R¹ is C₂-C₄-alkyl,
R² is C₂-C₄-alkyl,
R³ is H or methyl,
R⁴ is H or methyl,
R⁵ and R⁶ together form a bridge having four carbon atoms unless B and D are CH₂ or CH, in which case B, D, R⁵ and R⁶ together preferably form an unsubstituted benzene fragment,
X is BF₄.

Ligands of the general formula (I) that are particularly preferred in accordance with the invention are 4-carboxy-N-(1,3-diisopropyl-1,3-dihydro-2H-benzo[d]imidazol-2-ylidene)-2,6-dimethylbenzenaminium tetrafluoroborate or 4-carboxy-N-(1,3-di-tert-butylimidazolidin-2-ylidene)benzenaminium tetrafluoroborate.

The present invention further relates to a bismuth catalyst of the general formula (II)

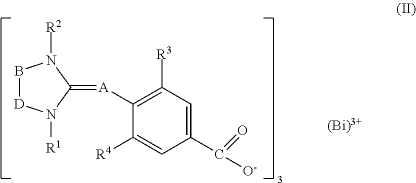

in which the variables are defined as follows:
A is N or (NH)⁺(X)⁻,
B is CH₂, CH, CR⁵ or CR⁵H,
D is CH₂, CH, CR⁶ or CR⁶H,
R¹ is H or C₁-C₁₀-alkyl,
R² is H or C₁-C₁₀-alkyl,
R³ is H or C₁-C₁₀-alkyl,
R⁴ is H or C₁-C₁₀-alkyl,
R⁵ is C₁-C₁₀-alkyl,
R⁶ is C₁-C₁₀-alkyl, or R⁵ and R⁶ together form a bridge having four carbon atoms,
where B, D, R⁵ and R⁶ may together form a benzene fragment that may optionally be at least monosubstituted by one R⁷ radical,
R⁷ is C₁-C₁₀-alkyl,
X is BF₄, OH or halogen,
where, in each of the three ligands of the bismuth catalyst, the respective variables may be defined independently of the respective two other ligands, or all three ligands have the same definition.

However, it is preferable that all three ligands in the bismuth catalyst of the general formula (II) have the same definition.

The above definitions and preferences with regard to the ligand of the general formula (I) are analogously also applicable to the present bismuth catalyst of the general formula (II).

According to the invention, it is preferable that
i) B and D, and optionally R⁵ and R⁶, each have the same definition, where B, D, R⁵ and R⁶ together optionally form a benzene fragment which is preferably unsubstituted, and/or
ii) R¹ and R² each have the same definition, and/or
iii) R³ and R⁴ each have the same definition, and/or
iv) halogen is chlorine or bromine, if X is halogen.

It is likewise also possible here that R¹ and R² are different and/or R³ and R⁴ are different. It is of course also possible that B and D are different.

It is particularly preferable that, in the general formula (II),
A is (NH)⁺(X)⁻,
B is CH₂, CH, CR⁵ or CR⁵H,
D is CH₂, CH, CR⁶ or CR⁶H,
R¹ is C₂-C₄-alkyl,
R² is C₂-C₄-alkyl,
R³ is H or methyl,
R⁴ is H or methyl,
R⁵ and R⁶ together form a bridge having four carbon atoms unless B and D are CH₂ or CH, in which case B, D, R⁵ and R⁶ together preferably form an unsubstituted benzene fragment,
X is BF₄.

The bismuth catalyst of the general formula (II) is more preferably bismuth (III) (4-((1,3-di-tert-butylimidazolidin-2-ylidene)ammonio)benzoate)tetrafluoroborate) 3 ([Bi(O₂C(PNsItBu)H)₃][BF₄]₃).

However, it is also possible that the central atom used in the catalyst of the invention, rather than a central bismuth atom, is a different central metal atom, for example a central zinc, titanium or zirconium atom.

Therefore, the present invention also further provides a metal catalyst which is prepared by reacting at least one compound of the general formula (I) with at least one metal compound, where the metal compound is preferably a bismuth, zinc, titanium or zirconium compound, preferably a bismuth compound.

The present invention further relates to a precursor of a ligand of the general formula (III)

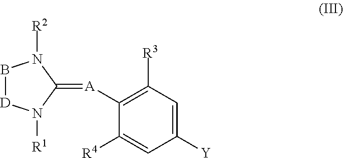

in which the variables are defined as follows:
A is N or (NH)⁺(X)⁻,
B is CH₂, CH, CR⁵ or CR⁵H,
D is CH₂, CH, CR⁶ or CR⁶H,
R¹ is H or C₁-C₁₀-alkyl,
R² is H or C₁-C₁₀-alkyl,
R³ is H or C₁-C₁₀-alkyl,
R⁴ is H or C₁-C₁₀-alkyl,
R⁵ is C₁-C₁₀-alkyl,
R⁶ is C₁-C₁₀-alkyl, or R⁵ and R⁶ together form a bridge having four carbon atoms,
where B, D, R⁵ and R⁶ may together form a benzene fragment that may optionally be at least monosubstituted by one R⁷ radical,
R⁷ is C₁-C₁₀-alkyl,
X is BF₄, OH or halogen,
Y is halogen, preferably Br.
In the general formula (III), it is preferably the case that
A is (NH)⁺(X)⁻,
B is CH₂, CH, CR⁵ or CR⁵H,
D is CH₂, CH, CR⁶ or CR⁶H,
R¹ is C₂-C₄-alkyl,
R² is C₂-C₄-alkyl,
R³ is H or methyl,
R⁴ is H or methyl,
R⁵ and R⁶ together form a bridge having four carbon atoms unless B and D are CH₂ or CH, in which case B, D, R⁵ and R⁶ together preferably form an unsubstituted benzene fragment,
X is BF₄.

The above definitions and preferences with regard to the ligand of the general formula (I) and with regard to the bismuth catalyst of the general formula (II) are also applicable analogously to the present precursor of a ligand of the general formula (III).

Most preferably, the precursor is N-(4-bromo-2,6-dimethylphenyl)-1,3-diisopropylbenzimidazol-2-imine (Br(P″NBiPr)) or N-(4-bromophenyl)-1,3-di-tert-butylimidazolidin-2-imine (Br(PNsItBu)).

The present invention also further provides a process for preparing an inventive ligand of the general formula (I), wherein the inventive ligand of the general formula (I) is obtained from the corresponding precursor of a ligand of the general formula (III) by carboxylation of said precursor in the presence of a catalyst.

A suitable catalyst for the carboxylation is, for example, n-butyllithium.

The present invention also further provides a process for preparing a bismuth catalyst of the general formula (II), wherein
i) at least one compound of the general formula (I) is reacted with
ii) at least one bismuth compound selected from Bi₂O₃, bismuth carbonate, bismuth hydrogencarbonate, bismuth halide, bismuth carboxylate, Bi(C₆-C₁₄-aryl)₃, Bi(C₁-C₁₂-alkyl)₃ and metallic bismuth.

In principle, it is possible in the process of the invention for preparing a bismuth catalyst of the general formula (II) to use any bismuth compound capable of forming the central bismuth atom in the inventive bismuth catalyst of the general formula (II) by reaction with the corresponding compound of the general formula (I). Bismuth compounds as such are known to those skilled in the art. If, in accordance with the invention, the bismuth compound used is a bismuth halide, it is preferably a chlorine compound, especially BiCl₃.

The bismuth compound is preferably selected from Bi₂O₃, BiCl₃, Bi(C₆H₅)₃ and metallic bismuth.

The inventive bismuth catalysts of the general formula (II) are preferably prepared by reacting at least one compound of the general formula (I) with at least one bismuth compound, wherein
i) the reaction is conducted under a protective atmosphere and/or in the presence of at least one solvent, especially toluene, acetonitrile or tetrahydrofuran, and/or ii) the reaction is conducted for at least 10 hours and/or at a temperature of at least 100° C., and/or
iii) after the reaction, volatile constituents are removed, the bismuth catalyst is dried under reduced pressure and/or a recrystallization is carried out.

The present invention further provides for the use of at least one bismuth catalyst according to the definitions above for preparation of compounds comprising a urethane group.

A further embodiment of the present invention encompasses a ligand of the general formula (I)

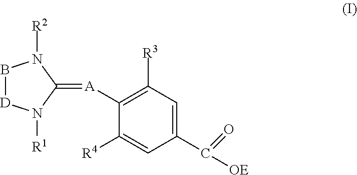

in which the variables are defined as follows:
A is N or (NH)⁺(X)⁻,
B is CH₂, CH, CR⁵ or CR⁵H,
D is CH₂, CH, CR⁶ or CR⁶H,
E is H or alkali metal,
R¹ is H or C₁-C₁₀-alkyl,
R² is H or C₁-C₁₀-alkyl,
R³ is H or C₁-C₁₀-alkyl,
R⁴ is H or C₁-C₁₀-alkyl,
R⁵ is C₁-C₁₀-alkyl,
R⁶ is C₁-C₁₀-alkyl, or R⁵ and R⁶ together form a bridge having four carbon atoms,
where B, D, R⁵ and R⁶ may together form a benzene fragment that may optionally be at least monosubstituted by one R⁷ radical,
R⁷ is C₁-C₁₀-alkyl,
X is BF₄, OH or halogen.

The invention is illustrated hereinafter by examples.

### I. Synthesis of the Electron-Rich Carboxylates and Comparative Examples

### N-(4-Bromo-2,6-dimethylphenyl)-1,3-diisopropylbenzimidazol-2-imine (Br(P″NBiPr))

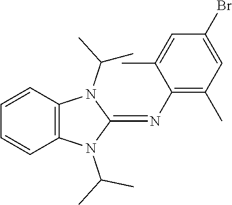 [SMILES: Cc1cc(Br)cc(C)c1N=c1n(C(C)C)c2ccccc2n1C(C)C]

The 2-chloro-1,3-diisopropylbenzimidazolium tetrafluoroborate precursor was synthesized by the following literature method: L. F. B. Wilm, T. Eder, C. Mück-Lichtenfeld, P. Mehlmann, M. Wünsche, F. Buβ and F. Dielmann, Green Chem., 2019, 21, 640-648.

In a Schlenk flask, 2-chloro-1,3-diisopropylbenzimidazolium tetrafluoroborate (3.38 g, 10.4 mmol, 1.00 eq.) and 4-bromo-2,6-xylidine (2.08 g, 10.4 mmol, 1.00 eq.) were dissolved in 35 ml of MeCN. After addition of Et₃N (2.90 ml, 20.8 mmol, 2.00 eq.), the reaction mixture was heated at 100° C. for 3 d. The progress of the reaction was monitored by 1H NMR spectroscopy. Subsequently, the solvent was removed under reduced pressure, and the residue was extracted with hot n-hexane (50 ml). The solution was stored at −20° C. overnight, and the Br(P NBiPr) product was obtained as a crystalline white solid. Subsequently, the Br(P″NBiPr) product was dried under reduced pressure at 80° C. for 24 h. Yield: 89% (3.90 g, 9.74 mmol).

### N-(4-Bromophenyl)-1,3-di-tert-butylimidazolidin-2-imine (Br(PNsItBu); Catalyst According to Comparative Example Comp. 2)

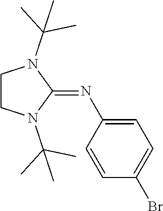 [SMILES: CC(C)(C)N1CCN(C(C)(C)C)C1=Nc1ccc(Br)cc1]

The 2-chloro-1,3-di-tert-butylimidazolium chloride precursor was likewise synthesized by the above literature method.

In a Schlenk flask, 2-chloro-1,3-di-tert-butylimidazolium chloride (12.5 g, 49.5 mmol, 1.00 eq.) and p-bromoaniline (8.52 g, 49.5 mmol, 1.00 eq.) were dissolved in 150 ml of MeCN. After addition of Et₃N (13.8 ml, 99.0 mmol, 2.00 eq.), the reaction mixture was heated at 80° C. for 4 d. The progress of the reaction was monitored by 1H NMR spectroscopy. Subsequently, the solvent was removed under reduced pressure, and the residue was extracted with hot n-hexane (150 ml). The solution was stored at −20° C. overnight, and the Br(PNsItBu) product was obtained as a crystalline yellow solid. Subsequently, the Br(PNsItBu) product was dried under reduced pressure at 80° C. for 24 h. Yield: 87% (15.1 g, 42.9 mmol).

### 4-carboxy-N-(1,3-diisopropyl-1,3-dihydro-2H-benzo[d]imidazol-2-ylidene)-2,6-dimethylbenzenaminium tetrafluoroborate ([HO₂C(P′NBiPr)H][BF₄])

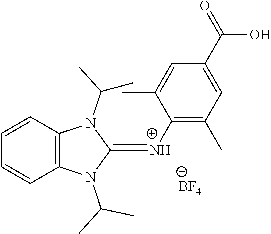

In a Schlenk flask, Br(P NBiPr) (2.18 g, 5.44 mmol, 1.00 eq.) was dissolved in 25 ml of THF and cooled to −78° C. Subsequently, a 1.6 M solution of n-butyllithium in n-hexane (3.40 ml, 5.44 mmol, 1.00 eq.) was slowly added dropwise, and the mixture was stirred at this temperature for 1 h. Subsequently, CO₂ was passed through the reaction solution with a Teflon cannula for 15 min, followed by stirring at room temperature for 1 h. All volatile constituents were removed under reduced pressure, and 10 ml of water was added to the residue. Subsequently, a 5.5 M solution of tetrafluoroboric acid in water (2.00 ml, 10.9 mmol, 2.00 eq.) was added gradually, and the mixture was stirred for 15 min. The [HO₂C(P″NBiPr)H][BF₄] product precipitates out as a white solid. This was filtered, and washed first with water and then with Et₂O (3×20 ml). Finally, [HO₂C(P″NBiPr)H] [BF₄] was dried under reduced pressure at 120° C. for 24 h, and obtained as a white solid with a yield of 84% (2.10 g, 4.6 mmol).

### 4-((1,3-diisopropyl-1,3-dihydro-2H-benzo[d]imidazol-2-ylidene)amino)-3,5-dimethylbenzoic acid (HO₂C(P″NBiPr))

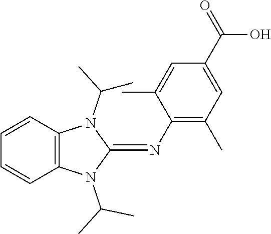 [SMILES: Cc1cc(C(=O)O)cc(C)c1N=c1n(C(C)C)c2ccccc2n1C(C)C]

A Schlenk flask was initially charged with [HO₂C(P″NBiPr)H][BF₄] (340 mg, 0.75 mmol) and sodium tert-butoxide (72.0 mg, 0.75 mmol). After addition of 5 ml of tetrahydrofuran, the reaction solution was stirred at room temperature for 1 h. Subsequently, the suspension was filtered, and the solution was blanketed with n-hexane and stored overnight. The crystalline compound HO₂C(P″NBiPr) was washed with the mother liquor and dried under reduced pressure at room temperature overnight. Yield: 93% (256 mg, 0.70 mmol).

### 4-carboxy-N-(1,3-di-tert-butylimidazolidin-2-ylidene)benzenaminium tetrafluoroborate ([HO₂C(PNsItBu)H] [BF₄])

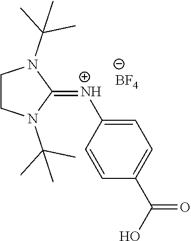

In a Schlenk flask, Br(PNsItBu) (5.64 g, 16.0 mmol, 1.00 eq.) was dissolved in 50 ml of THF and cooled to −78° C. Subsequently, a 1.6 M solution of n-butyllithium in n-hexane (10.0 ml, 16.0 mmol, 1.00 eq.) was slowly added dropwise, and the mixture was stirred at this temperature for 1 h. Subsequently, CO₂ was passed through the reaction solution with a Teflon cannula for 15 min, followed by stirring at room temperature for 1 h. All volatile constituents were removed under reduced pressure, and 25 ml of water was added to the residue. Subsequently, a 5.5 M solution of tetrafluoroboric acid in water (5.90 ml, 32.0 mmol, 2.00 eq.) was added gradually, and the mixture was stirred for 15 min. The [HO₂C(PNsItBu)H] [BF₄] product precipitates out as a white solid. This was filtered, and washed first with water and then with Et₂O (3×50 ml). Finally, [HO₂C(PNsItBu)H] [BF₄] was dried under reduced pressure at 120° C. for 24 h, and obtained as a white solid with a yield of 90% (5.90 g, 14.6 mmol).

### 4-((1,3-di-tert-butylimidazolidin-2-ylidene)amino)benzoic acid (HO₂C(PNsItBu))

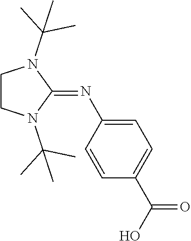 [SMILES: CC(C)(C)N1CCN(C(C)(C)C)C1=Nc1ccc(C(=O)O)cc1]

A Schlenk flask was initially charged with [HO₂C(PNsItBu)H] [BF₄] (304 mg, 0.75 mmol) and sodium tert-butoxide (72.0 mg, 0.75 mmol). After addition of 5 ml of tetrahydrofuran, the reaction solution was stirred at room temperature for 1 h. Subsequently, the suspension was filtered, and the solution was blanketed with n-hexane and stored overnight. The crystalline compound HO₂C(PNsItBu) was washed with the mother liquor and dried under reduced pressure at room temperature overnight. Yield: 92% (219 mg, 0.69 mmol).

### Bismuth (III) (4-((1,3-di-tert-butylimidazolidin-2-ylidene)ammonio)benzoate)tetrafluoroborate)₃ ([Bi(O₂C(PNsItBu)H)₃] [BF₄]₃/Bi(O₂C(PNsItBu))₃; Catalyst According to Working Example Ex. 4)

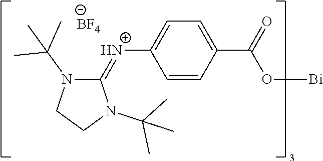

The compound [Bi(O₂C(PNsItBu)H)₃][BF₄]₃ was prepared at 80° C. by a modified literature method (P. C. Andrews, G. B. Deacon, P. C. Junk, I. Kumar and M. Silberstein, Dalton Trans., 2006, 4852-4858) in 20 ml of acetonitrile using BiPh₃ (88.0 mg, 0.20 mmol) and the compound [HO₂C(PNsItBu)H] [BF₄] (243 mg, 0.60 mmol). Subsequently, the solids were washed with 3×5 ml of toluene and dried under reduced pressure at 120° C. overnight. Yield: 95% (270 mg, 0.19 mmol).

The catalyst Bi(O₂C(PNsItBu))₃ was prepared by preparing the amount required for catalysis by deprotonation of [Bi(O₂C(PNsItBu)H)₃][BF₄]₃ with a standard potassium tert-butoxide solution in tetrahydrofuran, and using it directly for the urethane reaction.

### Bismuth 4-butylbenzoate (Bi(bbz)₃; Catalyst According to Comparative Example Comp. 1)

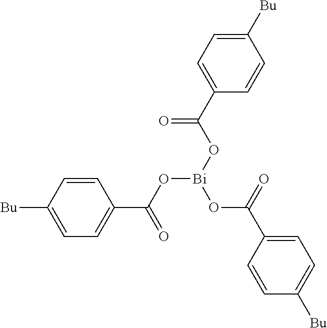

The compound Bi(bbz)₃ was prepared in quantitative yield by a modified literature method (P. C. Andrews, G. B. Deacon, P. C. Junk, I. Kumar and M. Silberstein, Dalton Trans., 2006, 4852-4858) using BiPh₃ (440 mg, 1.00 mmol) and 4-butylbenzoic acid (540 mg, 3.00 mmol).

### II. Determination of Catalyst Activity

The respective catalyst activity of the individual working and comparative examples can be found in table 1 below. Catalyst activity is tested by means of a reaction in which a compound comprising a urethane group is formed. For this purpose, 11 mmol of 2-ethylhexyl (6-isocyanatohexyl) carbamate (commercially available as Desmodur LD (3.3 ml)) as reactant is reacted with 11 mmol of n-butanol (1 ml). The reaction was conducted at room temperature in the presence of a solvent (2 ml of xylene) and of the catalysts listed in table 1 (with a content of 0.05 mol % of catalyst based on the amount of bismuth).

The decrease in isocyanate and hence the formation of a urethane group are examined by horizontal ATR-IR spectroscopy. For this purpose, 0.05 ml is withdrawn from the reaction solution at defined time intervals and examined directly by spectroscopy. The conversion is determined by the relative decrease in intensity of the asymmetric isocyanate stretching vibration at 2250-2285 cm⁻¹. The starting content of free isocyanate was determined at room temperature of the reaction solution in the absence of any catalyst. All IR spectra were normalized to the bands of the symmetric and asymmetric stretching vibrations of the CH₂ groups (3000-2870 cm-1).

The catalysts used are detailed in table 1:
Comparative example 1 (comp. 1): Bi(bbz)₃
Comparative example 2 (comp. 2): Br(PNsItBu)
Comparative example 3 (comp. 3): Bi(bbz)₃+3 eq. Br(PNsItBu)
Example 4 (ex. 4): Bi(O₂C(PNsItBu))₃
Comparative example 5 (comp. 5): no catalyst

TABLE 1

| | | | | | Bi(bbz)₃ + | | | | |
|---|---|---|---|---|---|---|---|---|---|
| | | | | | 3 eq. | | | | |
| | Bi(bbz)₃ | | Br(PNsltBu) | | Br(PNsltBu) | | Bi(O₂C(PNsltBu))₃ | | no catalyst |
| Comp. 1 | NCO | Comp. 2 | NCO | Comp. 3 | NCO | Ex. 4 | NCO | Comp. 5 | NCO |
| t/min | degradation | t/min | degradation/% | t/min | degradation/% | t/min | degradation/% | t/min | degradation/% |
| 0 | 0 | 0 | 0 | 0 | 0 | 0 | 0 | 0 | 0 |
| 5 | 1.0 | 10 | 3.5 | 10 | 6.2 | 5 | 4.7 | 20 | 6.0 |
| 10 | 2.0 | 20 | 7.2 | 20 | 10.3 | 10 | 7.5 | 40 | 8.8 |
| 15 | 3.6 | 40 | 9.7 | 40 | 15.8 | 15 | 16.3 | 60 | 9.5 |
| 20 | 5.1 | 60 | 11.8 | 60 | 18.7 | 20 | 35.5 | | |
| 25 | 6.2 | | | | | 25 | 57.2 | | |
| 30 | 8.2 | | | | | 30 | 73.3 | | |
| 40 | 10.5 | | | | | 40 | 85.4 | | |
| 50 | 13.5 | | | | | 50 | 89.7 | | |
| 60 | 14.7 | | | | | 60 | 92.9 | | |

As can be inferred from table 1, neither the bismuth carboxylate with the bbz ligand (comp. 1) nor the haloimine Br(PNsItBu) (comp. 2) shows elevated activity in the urethane reaction compared to the uncatalyzed reaction. Even the mixture consisting of Bi(bbz)₃ and 3 eq. Br(PNsItBu) (comp. 3) shows only a slight increase compared to the uncatalyzed reaction in terms of the catalytic activity of the urethane reaction. Surprisingly, the compound Bi(O₂C(PNsItBu))₃, in the first 10 minutes of the catalysis, is just as inactive as the mixture consisting of Bi(bbz)₃ and 3 eq. Br(PNsItBu) (comp. 3). After this inhibiting phase, however, a remarkable rise in catalytic activity is observed, which reaches a conversion of 90% after 60 min.

1. A ligand of formula (I) 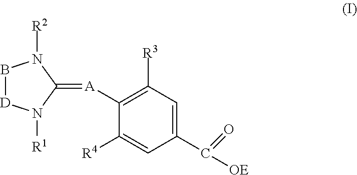
in which the variables are defined as follows:
A is N or (NH)⁺(X)⁻,
B is CH₂, CH, CR⁵ or CR⁵H,
D is CH₂, CH, CR⁶ or CR⁶H,
E is H or an alkali metal,
R¹ is H or a C₁-C₁₀-alkyl,
R² is H or a C₁-C₁₀-alkyl,
R³ is H or a C₁-C₁₀-alkyl,
R⁴ is H or a C₁-C₁₀-alkyl,
R⁵ is a C₁-C₁₀-alkyl,
R⁶ is a C₁-C₁₀-alkyl, or R⁵ and R⁶ together form a bridge having four carbon atoms,
B, D, R⁵ and R⁶ together optionally form a benzene fragment that is optionally at least monosubstituted by one R⁷ radical,
R⁷ is a C₁-C₁₀-alkyl, and
X is BF₄, OH or a halogen.

2. The ligand according to claim 1, wherein
i) B and D, and optionally R⁵ and R⁶, each have the same definition, where B, D, R⁵ and R⁶ together optionally form a benzene fragment which is optionally unsubstituted, and/or
ii) R¹ and R² each have the same definition, and/or
iii) R³ and R⁴ each have the same definition, and/or
iv) with the proviso that E is an alkali metal, the alkali metal is sodium or potassium, and/or
v) with the proviso that X is a halogen, the halogen is chlorine or bromine.

3. A ligand according to claim 1, wherein the ligand is 4-carboxy-N-(1,3-diisopropyl-1,3-dihydro-2H-benzo[d]imidazol-2-ylidene)-2,6-dimethylbenzenaminium tetrafluoroborate or 4-carboxy-N-(1,3-di-tert-butylimidazolidin-2-ylidene)benzenaminium tetrafluoroborate.

4. A bismuth catalyst of formula (II) 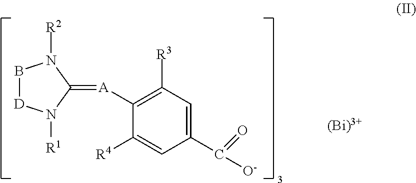
in which the variables are defined as follows:
A is N or (NH)⁺(X)⁻,
B is CH₂, CH, CR⁵ or CR⁵H,
D is CH₂, CH, CR⁶ or CR⁶H,
R¹ is H or a C₁-C₁₀-alkyl,
R² is H or a C₁-C₁₀-alkyl,
R³ is H or a C₁-C₁₀-alkyl,
R⁴ is H or a C₁-C₁₀-alkyl,
R⁵ is a C₁-C₁₀-alkyl,
R⁶ is a C₁-C₁₀-alkyl, or R⁵ and R⁶ together form a bridge having four carbon atoms,
B, D, R⁵ and R⁶ together optionally form a benzene fragment that is optionally at least monosubstituted by one R⁷ radical,
R⁷ is a C₁-C₁₀-alkyl, and
X is BF₄, OH or a halogen,
wherein
all the three ligands of the bismuth catalyst are the same or the respective variables in each of the three ligands are defined independently of the respective other ligands, and
a double bond rather than a single bond is optionally present between B and D.

5. The bismuth catalyst according to claim 4, wherein all the three ligands of the bismuth catalyst are the same.

6. The bismuth catalyst according to claim 4, wherein
i) B and D, and optionally R⁵ and R⁶, each have the same definition, where B, D, R⁵ and R⁶ together optionally form a benzene fragment which is optionally unsubstituted, and/or
ii) R¹ and R² each have the same definition, and/or
iii) R³ and R⁴ each have the same definition, and/or
iv) with the proviso that X is a halogen, the halogen is is chlorine or bromine.

7. The bismuth catalyst according to claim 4, wherein
A is (NH)⁺(X)⁻,
R¹ is a C₂-C₄-alkyl,
R² is a C₂-C₄-alkyl,
R³ is H or methyl,
R⁴ is H or methyl,
R⁵ and R⁶ together form a bridge having four carbon atoms unless B and D are CH₂ or CH, in which case B, D, R⁵ and R⁶ together optionally form an unsubstituted benzene fragment, and
X is BF₄.

8. The bismuth catalyst according to claim 4, wherein the bismuth catalyst is bismuth (III) (4-((1,3-di-tert-butylimidazolidin-2-ylidene)ammonio)benzoate)tetrafluoroborate) 3 ([Bi(O₂C(PNsItBu)H)₃][BF₄]₃).

9. A precursor of a ligand of formula (III) 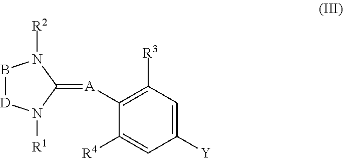
in which the variables are defined as follows:
A is N or (NH)⁺(X)⁻,
B is CH₂, CH, CR⁵ or CR⁵H,
D is CH₂, CH, CR⁶ or CR⁶H,
R¹ is H or a C₁-C₁₀-alkyl,
R³ is H or a C₁-C₁₀-alkyl,
R⁴ is H or a C₁-C₁₀-alkyl,
R⁵ is a C₁-C₁₀-alkyl,
R⁶ is a C₁-C₁₀-alkyl, or R⁵ and R⁶ together form a bridge having four carbon atoms,
B, D, R⁵ and R⁶ together optionally form a benzene fragment that is optionally at least monosubstituted by one R⁷ radical,
R⁷ is a C₁-C₁₀-alkyl,
X is BF₄, OH or a halogen, and
Y is a halogen.

10. A process for preparing a ligand, the process comprising:
subjecting the precursor of a ligand of the formula (III) according to claim 9 to carboxylation in the presence of a catalyst.

11. A process for preparing the bismuth catalyst of the formula (II) according to claim 4 the process comprising: reacting
i) at least one compound of formula (I) with
ii) at least one bismuth compound selected from the group consisting of Bi₂O₃, bismuth carbonate, bismuth hydrogencarbonate, a bismuth halide, a bismuth carboxylate, Bi(C₆-C₁₄-aryl)₃, Bi(C₁-C₁₂-alkyl)₃ and metallic bismuth; 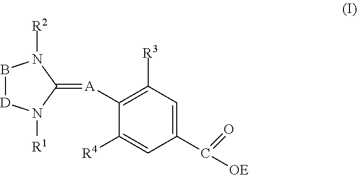
wherein E is H or an alkali metal, and all the other variables in the formula (I) have the same definitions as the corresponding variables in the formula (II).

12. The process according to claim 11, wherein the at least one bismuth-containing compound is selected from the group consisting of Bi₂O₃, BiCl₃, Bi(C₆H₅)₃ and metallic bismuth.

13. The process according to claim 11, wherein
i) the reaction is conducted under a protective atmosphere and/or in the presence of at least one solvent, and/or
ii) the reaction is conducted for at least 10 hours and/or at a temperature of at least 100° C., and/or
iii) after the reaction, volatile constituents are removed, the bismuth catalyst is dried under reduced pressure and/or a recrystallization is carried out.

14. A process for preparing a compound comprising a urethane group, the process comprising:
preparing the compound in the presence of at least one bismuth catalyst according to claim 4.

15. The ligand according to claim 1, wherein
A is (NH)⁺(X)⁻,
E is H,
R¹ is a C₂-C₄-alkyl,
R² is a C₂-C₄-alkyl,
R³ is H or methyl,
R⁴ is H or methyl,
R⁵ and R⁶ together form a bridge having four carbon atoms unless B and D are CH₂ or CH, in which case B, D, R⁵ and R⁶ together optionally form an unsubstituted benzene fragment, and
X is BF₄.

16. The process according to claim 13, wherein the reaction is conducted in the presence of at least one solvent selected from the group consisting of toluene, acetonitrile, and tetrahydrofuran.
